(19) United States
(12) Patent Application Publication
(10) Pub. No.: US 2015/0018865 A1
(43) Pub. Date: Jan. 15, 2015

(54) TATTOO NEEDLE GUIDE TUBE WITH CURVED THROUGH HOLE

(71) Applicant: Mei-Chi-Na Hsinyen Co., Ltd., Taipei City (TW)

(72) Inventor: Su-Lin LIN, Taipei City (TW)

(21) Appl. No.: 13/935,623

(22) Filed: Jul. 5, 2013

(51) Int. Cl. A61M 37/00

(52) U.S. Cl. CPC A61M 37/0076
USPC 606/186

(57) ABSTRACT

The present invention is to provide a tattoo needle guide tube, which has a tubular body defining a receiving space therein, a first through hole at a top end thereof and a second through hole at a bottom end thereof. Both through holes are in communication with the receiving space, and the outer periphery of the top end can be connected to a main body of an eyebrow tattoo machine, so as to allow a plurality of eyebrow tattoo needles in the main body to pass sequentially through the first through hole and receiving space and have needle tips exposed outside the second through hole. The tattoo needle guide tube is characterized in that the second through hole has a curved shape and is configured and sized to render the needle tips into a curved arrangement, so as for an eyebrow tattooist to make desired hair-like tattoos with ease.

### FIELD OF THE INVENTION

The present invention relates to a tattoo needle guide tube, more particularly to a tattoo needle guide tube allowing a plurality of eyebrow tattoo needles in a main body of an eyebrow tattoo machine to pass therethrough and have needle tips exposed outside the tattoo needle guide tube in a curved arrangement, so as for an eyebrow tattooist to make desired hair-like tattoos with ease.

### BACKGROUND OF THE INVENTION

Eyebrow tattooing is a skin coloring procedure involving wounds deliberately caused to the skin. More specifically, a pigment is inserted into skin tissues to form a stable colored area on the skin. With the advancement of eyebrow tattooing techniques, and with more and more people desiring eyebrow tattoos, eyebrow tattooing has evolved from the single-needle mode to the flat-shader mode, among others. A flat shader consists of a row of aligned needles which work in unison during tattooing.

Eyebrow tattooing techniques in current use are briefly described as follows:

(1) “Planar eyebrow tattoos” are based on standard eyebrow shapes and have a uniform distribution of color over the tattooed area. These eyebrow tattoos look as if they were painted only a moment ago.

(2) “Simulated three-dimensional eyebrow tattoos” are typically made with a flat shader, which can be maneuvered in different ways so that the resultant tattoos look like having rows of hair growing in three dimensions. In addition, dyes of multiple colors are used to provide color differences that give the eyebrow tattoos a more three-dimensional and hence more natural look.

(3) “Three-dimensional eyebrow tattoos” are also created with a flat shader, but the pigment used is of one color only. Tiny tattoo points of progressively darker shades are made to produce a three-dimensional effect.

(4) “Dotted eyebrow tattoos” are applied by piercing and dying eyebrow hair follicles with a needle coated with a liquid preparation. This tattooing technique, though slightly more painful than the above three, can make a beautiful pair of eyebrow tattoos whose thicknesses vary in exactly the same way as natural eyebrows.

It can be known from the above that flat shaders nowadays are in extensive use to produce more three-dimensional- and natural-looking eyebrow tattoos. Because of that, eyebrow tattoo machines for use with flat shaders have also been improved to enable higher-quality and more convenient eyebrow tattooing. Generally speaking, an existing eyebrow tattoo machine includes a main body in which a transmission mechanism is provided. The transmission mechanism can drive a flat shader (i.e., a plurality of eyebrow tattoo needles) into reciprocal up-and-down motion along the central axis of the eyebrow tattoo machine. Hence, by operating the eyebrow tattoo machine, an eyebrow tattooist can apply eyebrow tattoos of predetermined shapes to a client's face.

However, while most people's eyebrows extend along a slightly curved surface, the needles in a commercially available flat shader are always arranged in a straight line. An eyebrow tattooist, therefore, must change his or her hand posture constantly in order to produce natural-looking eyebrow tattoos with a flat shader. Moreover, as eyebrow tattoos are popular mostly among females, who generally prefer thin eyebrows, an eyebrow tattooist wishing to achieve narrow eyebrow tattoos must move the eyebrow tattoo machine very slightly during the tattooing process, and the tattooed spot must be changed on a regular basis to give the resultant tattoos a three-dimensional and natural look. If the above operation continues for a long time, the eyebrow tattooist's hand must be overloaded, which may eventually turn into vocational injury.

The issue to be addressed by the present invention is to design a novel structure which can solve the aforementioned problems without having to substantially change the structure of a conventional eyebrow tattoo machine, and which therefore saves the cost otherwise required for replacing the entire eyebrow tattoo machine.

### BRIEF SUMMARY OF THE INVENTION

As stated above, a conventional eyebrow tattoo machine for use with flat shaders is typically designed for accommodating a plurality of linearly arranged eyebrow tattoo needles, but such a needle configuration tends to burden an eyebrow tattooist's hand and requires the flat shader in use be continually adjusted in position. In view of this, and in order to solve the foregoing problems without significant modification of the existing eyebrow tattoo machine structure, the inventor of the present invention conducted extensive research and experiment and finally succeeded in developing a tattoo needle guide tube with a curved through hole. It is hoped that the structure of this tattoo needle guide tube provides an effective solution to the aforesaid problems.

The present invention provides a tattoo needle guide tube having a curved through hole. The tattoo needle guide tube is connectable to an eyebrow tattoo machine and has a tubular body defining a receiving space therein. The tattoo needle guide tube also has a first through hole at the top end and a second through hole at the bottom end. Both through holes are in communication with the receiving space. The outer periphery of the top end of the tattoo needle guide tube can be connected to a main body of the eyebrow tattoo machine, allowing a plurality of eyebrow tattoo needles in the main body to pass sequentially through the first through hole and the receiving space and have their needle tips exposed outside the second through hole. The tattoo needle guide tube is characterized in that the first through hole has a greater cross-sectional area than the second through hole and that the second through hole has a curved shape and is configured and sized to render the needle tips of the eyebrow tattoo needles into a curved arrangement outside the second through hole. The second through hole of the tattoo needle guide tube is so designed that an eyebrow tattooist can make the desired hair-like tattoos with ease, and convenience of use is thus greatly increased.

### BRIEF DESCRIPTION OF THE SEVERAL VIEWS OF THE DRAWINGS

The objects as well as the technical features and effects of the present invention will be best understood by referring to the following detailed description of some illustrative embodiments in conjunction with the accompanying drawings, in which:

FIG. 1 is a perspective view of an eyebrow tattoo machine employing the present invention;

FIG. 2 is a perspective view of a tattoo needle guide tube according to the present invention; and

FIG. 3 is a perspective view of the tattoo needle guide tube illustrated in FIG. 2 and a plurality of eyebrow tattoo needles extending therethrough.

### DETAILED DESCRIPTION OF THE INVENTION

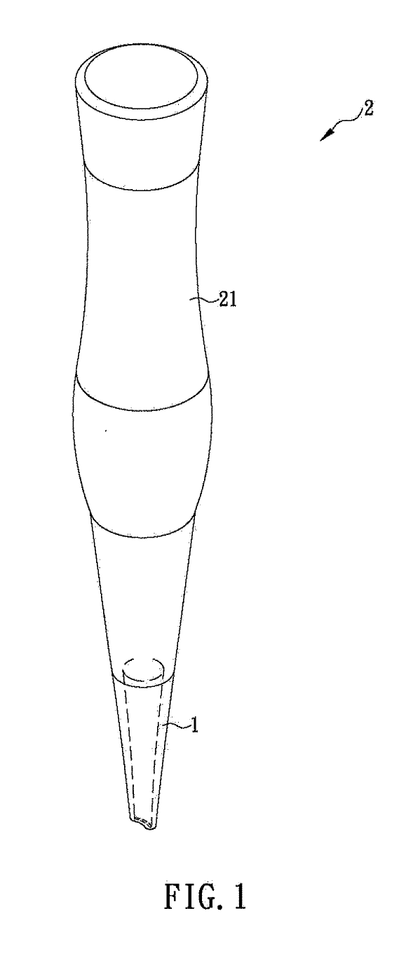

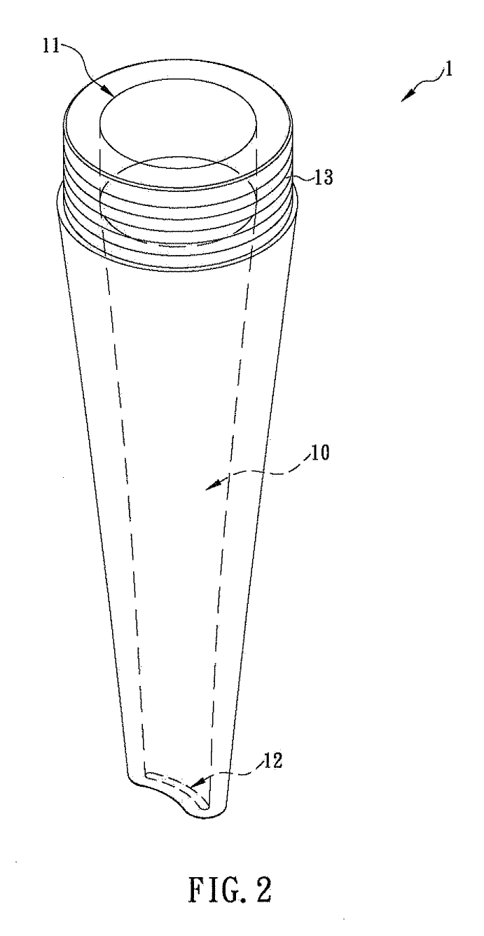

The present invention provides a tattoo needle guide tube having a curved through hole. Referring to FIG. 1 and FIG. 2, the tattoo needle guide tube 1 is to be connected to an eyebrow tattoo machine 2. In this embodiment, the tattoo needle guide tube 1 is a tubular body and is provided therein with a receiving space 10. The tattoo needle guide tube 1 is formed with a first through hole 11 at the top end and a second through hole 12 at the bottom end. The first through hole 11 and the second through hole 12 are in communication with the receiving space 10. The first through hole 11 has a greater cross-sectional area than the second through hole 12, and the second through hole 12 has a curved shape. The outer periphery of the top end of the tattoo needle guide tube 1 is provided with a connecting unit 13 (e.g., a screw thread, a projecting block, a groove, or a threaded hole) by which the tattoo needle guide tube 1 can be connected to a main body 21 of the eyebrow tattoo machine 2. The internal structure of the eyebrow tattoo machine 2 will not be detailed herein, given the fact that it is well known in the art and does not constitute a major technical feature of the present invention.

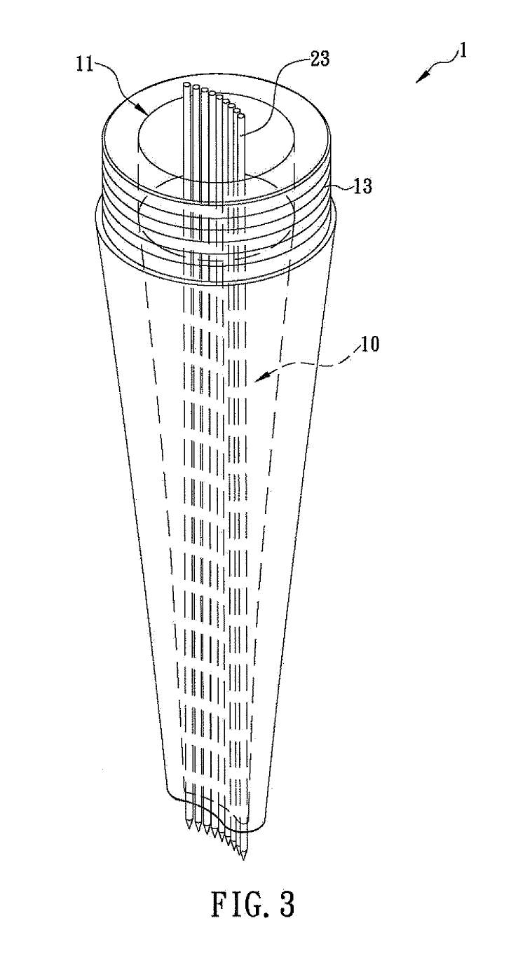

Referring to FIG. 1˜FIG. 3, a plurality of eyebrow tattoo needles 23 (in the form of a flat shader) are provided in the main body 21. The eyebrow tattoo needles 23 can be driven reciprocally up and down by a transmission mechanism (not shown) in the main body 21 such that the eyebrow tattoo needles 23 pass sequentially through the first through hole 11 and the receiving space 10 and have their needle tips exposed outside the second through hole 12. The ends of the eyebrow tattoo needles 23 that face away from the needle tips are connected to the transmission mechanism, but the eyebrow tattoo needles 23 themselves are not fixed to one another, meaning that each two adjacent eyebrow tattoo needles 23 can be spaced apart from each other to form a gap. When the needle tips of the eyebrow tattoo needles 23 are moved out of the second through holes 12, the eyebrow tattoo needles 23 slide along an inner wall portion of the tattoo needle guide tube 1 that is adjacent to the second through hole 12. As the second through hole 12 is curved, the needle tips of the eyebrow tattoo needles 23 are limited by the configuration and size of the second through hole 12 when sliding out of the second through hole 12. Consequently, gaps are formed between the needle tips of the eyebrow tattoo needles 23, preventing the needle tips from entering an orderly linear arrangement. The needle tips of the eyebrow tattoo needles 23 will end up in a curved arrangement similar to the curved configuration of the second through hole 12.

According to the above, a conventional eyebrow tattoo machine can employ the present invention without undergoing a major structural change. All that needs to be done is to replace the conventional tattoo needle guide tube with the one disclosed herein. The tattoo needle guide tube of the present invention allows the exposed needle tips of a plurality of eyebrow tattoo needles to be arranged in a curved manner, making it easy to create hair-like tattoos. More particularly, an eyebrow tattooist does not have to fine-tune his or her hand posture repeatedly as is conventionally required, and the workload of tattooing is thus substantially reduced. Furthermore, the present invention effectively increases convenience of use.

While the invention herein disclosed has been described by means of specific embodiments, numerous modifications and variations could be made thereto by those skilled in the art without departing from the scope of the invention set forth in the claims.

What is claimed is:

1. A tattoo needle guide tube with a curved through hole, wherein the tattoo needle guide tube is a tubular body connectable to an eyebrow tattoo machine and is provided therein with a receiving space, the tattoo needle guide tube having a top end formed with a first though hole and a bottom end formed with a second through hole, both said through holes being in communication with the receiving space, the top end of the tattoo needle guide tube having an outer periphery connectable to a main body of the eyebrow tattoo machine such that a plurality of eyebrow tattoo needles in the main body pass sequentially through the first through hole and the receiving space and have needle tips thereof exposed outside the second through hole, the tattoo needle guide tube being characterized in that:
the second through hole has a curved shape and is so configured and sized that the needle tips of the eyebrow tattoo needles are arranged in a curved manner while exposed outside the second through hole.

2. The tattoo needle guide tube of claim 1, wherein the first through hole has a greater cross-sectional area than the second through hole.

3. The tattoo needle guide tube of claim 2, wherein the outer periphery of the top end of the tattoo needle guide tube is provided with a connecting unit in order to connect to the main body.

4. The tattoo needle guide tube of claim 3, wherein the connecting unit is a screw thread.

5. The tattoo needle guide tube of claim 3, wherein the connecting unit is a projecting block.

6. The tattoo needle guide tube of claim 3, wherein the connecting unit is a groove.

7. The tattoo needle guide tube of claim 3, wherein the connecting unit is a threaded hole.
